(19) United States
(12) Patent Application Publication
(10) Pub. No.: US 2009/0275848 A1
(43) Pub. Date: Nov. 5, 2009

(54) Cardiac Risk Assessment

(75) Inventors: Marina V. Brockway, Shoreview, MN (US); Brian P. Brockway, Shoreview, MN (US)

Correspondence Address: FISH & RICHARDSON P.C., P.O. BOX 1022, MINNEAPOLIS, MN, 55440-1022, US

(73) Assignee: TRANSOMA MEDICAL, INC., Saint Paul, MN (US)

(21) Appl. No.: 12/112,924

(22) Filed: Apr. 30, 2008

(51) Int. Cl. A61B 5/04; A61N 1/00

(52) U.S. Cl. 600/513; 607/60

(57) ABSTRACT

In a system for assessing cardiac risk of a patient, a measuring component measures a cardiac signal comprised of multiple cardiac cycles at multiple periods in time. A processing component calculates sensitivity of cardiac function to sympathetic drive at each of the periods in time from the measured cardiac signal. A risk identification component evaluates a trend of the sensitivity over time as an indicator of a degree of cardiac risk.

### TECHNICAL FIELD

This disclosure relates to assessing a patient's cardiac risk.

### BACKGROUND

In recent years, health care expenditures related to chronic cardiovascular disease have exceeded $400 billion per year in the United States. A contributor to the high costs of caring for patients suffering from chronic cardiovascular disease is a limited ability to identify which of multiple available therapies is appropriate for a given patient. For example, in an SCD-HeFT ICD trial, it was found that after four years, only 20% of patients implanted with an implantable cardiac defibrillator (ICD) had received an appropriate therapy from the device. The trial illustrates a deficiency regarding appropriate identification of patients at risk of sudden cardiac death (SCD). Results of this study also indicate that about fifteen patients must be implanted with ICDs in order to save one life, resulting in marginal cost effectiveness. It is further understood that about 50% of sudden cardiac death cases occur in patients with compromised cardiac function, but their hearts are not sick enough to be indicated for an ICD. An ability to monitor some subpopulation of these patients to identify risk of SCD would be valuable in identifying patients that could benefit from an ICD or other therapy to prevent SCD. In addition, it has been well documented that patient non-compliance with prescribed therapies is a significant contributor to the high cost of caring for these patients. For example, about half of heart failure hospitalizations can be attributed to patients' failure to comply with prescribed therapeutic regimens.

It is well known that patients with heart failure exhibit increased sympathetic drive to neurohormonal receptors in the heart and elsewhere. This increased sympathetic drive exerts an adverse effect on the cardiovascular system, and can lead to pump failure and lethal arrhythmias (SCD). In addition, heart failure patients may have increased sensitivity of the cardiovascular system to sympathetic drive, which may provoke a downward spiral in cardiovascular function deterioration. Medications such as beta blockers and ACE-inhibitors have been developed to block the action of the sympathetic nervous system, and have resulted in decreased sensitivity of the cardiovascular system to sympathetic drive. Sympathetic drive and the sensitivity of the myocardium to sympathetic drive play a role in the progression of heart failure and the risk of SCD. Further, compliance with medications prescribed to mitigate the adverse impact of sympathetic drive is important to maintain good patient outcomes and reduce the cost of caring for these patients.

### SUMMARY

A cardiac or cardiovascular signal may be measured and analyzed to assess a patient's cardiac risk profile and disease progression, or to provide insight into a therapy's effectiveness on managing the patient's cardiac substrate, or to assess patient compliance with a prescribed therapy regimen.

In a first general aspect, a system for assessing cardiac risk of a patient includes a measuring component that measures a cardiac signal comprised of multiple cardiac cycles at multiple periods in time. The system also includes a processing component that calculates sensitivity of cardiac function to sympathetic drive at each of the periods in time from the measured cardiac signal. The system further includes a risk identification component that evaluates a trend of the sensitivity over time as an indicator of a degree of cardiac risk.

In various implementations, the processing component may extract parameters related to cardiovascular function. The risk identification component may evaluate the trend by evaluating a plot of the sensitivity versus time, or by comparing the trend with a predetermined threshold. The measuring component, the processing component, and the risk identification component may reside in an implantable device, or the measuring component may reside in an implantable device while the processing component and the risk identification component may reside in an external device. The system may include a first telemetry component in the implantable device and a second telemetry component in the external device, where the first telemetry component transmits at least a portion of the cardiac signal for receipt by the second telemetry component. The cardiac function may be indicated by an alternans burden over a selected period of time, and the alternans burden may be determined from the cardiac signal. The alternans burden may be a repolarization altemans burden, a QRS altemans burden, or a mechanical alternans burden. The sensitivity of cardiac function may be calculated by computing a ratio of change in the altemans burden to change in a sympathetic tone marker. The sensitivity of cardiac function may be calculated as an onset value of a sympathetic tone marker at which a sustained elevation in the altemans burden appears. The cardiac function may be indicated by a hypertension burden over a selected period of time, the hypertension burden determined from the cardiac signal. The hypertension burden may be elevated diastolic blood pressure or elevated systolic blood pressure. Sympathetic drive may be estimated from a heart rate variability measurement determined from the cardiac signal or a heart rate increase measurement determined from the cardiac signal. The cardiac signal may be an electrocardiogram signal, a blood pressure signal, an impedance measurement, or an electrogram signal measured with an intracardiac lead. The processing component may further calculate a baroreflex sensitivity value from the measured cardiac signal. An indication of heart rate turbulence, determined from the cardiac signal, may indicate the baroreflex sensitivity value. Heart rate turbulence may be quantified by two parameters: turbulence onset (TO) and/or turbulence slope (TS). TO can reflect an amount of heart rate acceleration following a premature beat. TS can reflect a rate of heart rate deceleration that follows heart rate acceleration. The risk identification component may adjust the trend based on circadian variability, or may filter the trend to remove circadian variability and short term variability. The trend of the sensitivity over time may indicate a degree of cardiac risk that exceeds an expected degree of cardiac risk because of disease progression, patient non-compliance with a prescribed therapy, or because of insufficient therapy effectiveness.

In another general aspect, a method of monitoring effect of a cardiovascular therapy for a patient includes receiving physiologic signal data associated with multiple cardiac cycles. The method also includes calculating sensitivity of cardiovascular function to sympathetic drive using the received physiologic signal data, and trending the computed sensitivity over time. The method further includes comparing the sensitivity trend to an expected trend, where the expected trend is based on a previous condition of the patient and a prescribed therapy regimen that specifies the cardiovascular therapy.

In various implementations, the cardiovascular therapy may modulate the effect of the sympathetic tone on cardiac function. The cardiovascular therapy may modulate vascular tone.

Some implementations may include one or more of the following advantages: patient cardiac risk may be assessed over an extended time period in an ambulatory setting, where the patient is free to go about her daily activities; risk of a severe cardiac event may be preemptively determined, so that appropriate therapeutic or preventative measures may be taken to prevent the event or mitigate its impact; patient compliance with a prescribed therapy regimen may be assessed; therapy effectiveness may be determined, and adjustments can easily be made as appropriate.

The details of one or more implementations are set forth in the accompanying drawings and the description below. Other features, objects, and advantages will be apparent from the description and drawings, and from the claims.

### DESCRIPTION OF DRAWINGS

FIG. 1 is an exemplary system that includes an implantable device and external equipment that can be used to assess a cardiac risk profile of a patient.

FIG. 2 is a flow chart of an exemplary process that can be used to assess patient cardiac risk using cardiac signal data.

FIG. 3 is a flow chart of an exemplary process that can be used to calculate sensitivity of cardiac function to sympathetic drive.

FIG. 4 is another flow chart of an exemplary process that can be used to calculate sensitivity of cardiac function to sympathetic drive, and includes additional information as compared to the flow chart of FIG. 3.

FIGS. 5A, 5B, and 5C are exemplary charts of data trended over time.

FIG. 6 is a series of exemplary charts that can be used to assess a patient's cardiac risk profile.

FIGS. 7A and 7B are exemplary charts of T-wave alternans amplitude data versus heart rate.

FIG. 8 is a block diagram of exemplary devices that can be used to calculate sensitivity of cardiac function to sympathetic drive and evaluate a trend of the sensitivity over time.

FIG. 9 is a simplified block diagram of an exemplary implantable device.

FIG. 10 is a graph of an exemplary dependence of T-wave alternans on heart rate.

Like reference symbols in the various drawings indicate like elements.

### DETAILED DESCRIPTION

Described herein are methods, systems, and devices that can be used to assess or determine a degree of cardiac risk for a patient. A cardiac or cardiovascular signal, such as a physiologic signal that is influenced by the patient's cardiac cycle or by cardiac parameters, can be measured. Without limitation, the signal may be an electrical signal or a hemodynamic signal, and may be measured using implanted electrodes or sensors, or alternatively using external electrodes or sensors. Any appropriate number of electrodes or sensors may be used (e.g., one, two, three, four, etc.). In some implementations, a combination of internal and external electrodes, sensors, or some combination can be used to measure one or more such physiologic signals for use in evaluating a patient's cardiac risk profile.

Examples of such signals can include an electrocardiogram (ECG) signal, an electrogram (EGM) signal, a blood pressure signal, a blood flow signal, or a signal comprised of impedance measurements. The signal may be measured at various locations within or outside of the body, including within a heart chamber (e.g., left ventricle, right ventricle, left atrium, right atrium), at an implanted subcutaneous location outside of the heart (e.g., in a pectoral region), within a body vessel (e.g., within an artery or vein), within or across an organ, at an external location on the patient's skin, and others.

Some implementations may assess patient compliance with a prescribed therapy regimen, such as to determine if the patient is taking prescribed medications according to a prescribed schedule. In this fashion, it may be determined, for example, that the patient is not complying with the prescribed therapy regimen, perhaps because of adverse side effects associated with prescribed medications or the therapy regimen. In response, a health care professional, or the patient herself, may be alerted that the therapy regimen is not being followed, so that an appropriate response may be taken. For example, the patient may be counseled on the importance of following the therapy regimen and encouraged to do so in the future, or the therapy regimen may be modified as appropriate.

Some implementations may also be used to assess therapy effectiveness, and may in some cases be used to determine that an up-titration in a patient's prescribed medications may be advisable. In some implementations, it can be determined that a prescribed therapy is not having the anticipated beneficial therapeutic effect for the patient, and the therapy can be adjusted appropriately in response. In this fashion, a cardiac signal can be collected and analyzed to determine a therapy's effectiveness in managing the cardiac substrate for a patient, or to determine a patient's compliance with a therapy regimen. As such, the patient may enjoy an improved quality of life according to some implementations, as the patient's cardiac substrates may be more effectively managed because the patient's physicians may be better able to track progress under a therapy program or assess when a patient is at increased risk of a severe cardiac event. With data trended over time, a more accurate analysis of a patient's condition can be determined. By analyzing patient data collected over an extended period of time, such as over one or more weeks, the patient's condition may be monitored to determine if the patient's health has improved from using a particular therapy. This can assist a health care professional in tailoring medications, which may improve patient longevity.

In various implementations, the cardiac or cardiovascular signal may be measured at multiple periods in time, such as over several seconds, minutes, hours, days, weeks, months, or years, and each recording may include information corresponding to multiple cardiac cycles. Using the measured signal, sensitivity of cardiac function to sympathetic drive may be calculated for each of the periods, and the sensitivity may be trended over time. The trend of the sensitivity over time may be evaluated to determine an indicator of a degree of cardiac risk for the patient. In some implementations, changes in the trend over time may be used to establish a risk indicator value indicative of a patient's susceptibility to sudden cardiac death, for example. In some implementations, changes in the trend over time may be used to alert to disease worsening. Various statistical methods may be used in these determinations, as will be discussed in greater detail below.

In some implementations, the methods described herein may be implemented entirely within one or more implantable devices. In other implementations, portions or all of the methods may be implemented in one or more external (i.e., non-implanted) devices, and in some cases a portion of a method may be implemented in one or more implantable devices and a remaining portion of the method may be implemented in one or more external devices.

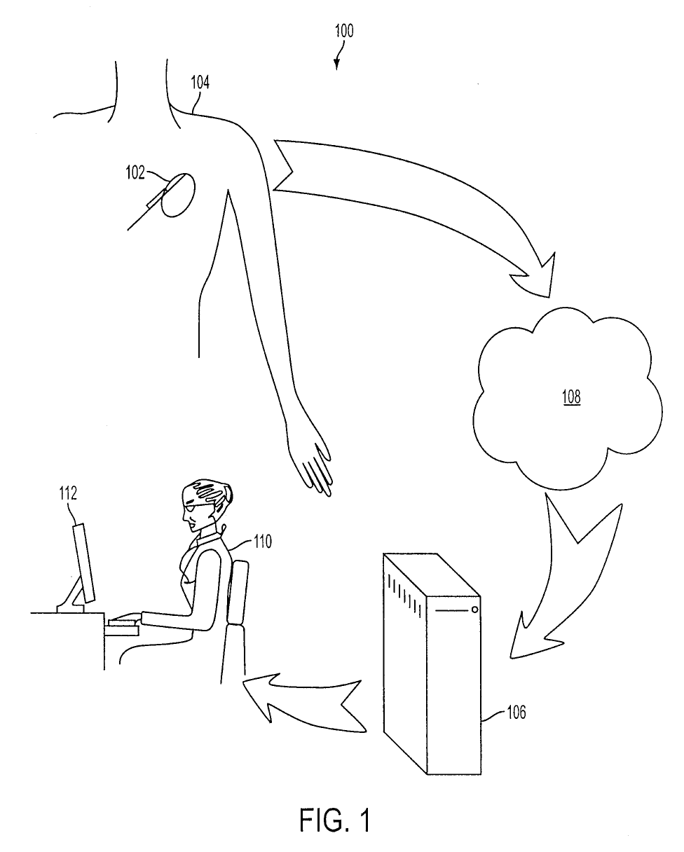

FIG. 1 is an exemplary system 100 that includes an implantable device 102 and external equipment that can be used to assess a cardiac risk profile of a patient 104. In this illustrative implementation, the implantable device 102 is shown implanted subcutaneously in a left chest region of the patient 102. In some implementations, the device 102 includes two or more electrodes for measuring physiologic electrical activity, such as an ECG or electrogram signal, or a body impedance. In some implementations, the device 102 includes a sensor for measuring a hemodynamic signal, such as a pressure sensor for measuring a body pressure within the patient or a blood flow sensor to measure blood flow through a body vessel. In some implementations, the device 102 can measure both an electrical signal and a hemodynamic signal, each of which can be used in assessing cardiac risk for the patient. The device 102 is depicted as a monitoring device, but in some implementations the device may include therapeutic functionality as well, or may be communicably connected to a separate implantable therapy device. Without limitation, such therapeutic functions or devices may include drug delivery or a drug pump, an implantable cardio-defibrillator device, a pacing device, including a device enabled for anti-tachycardia pacing, an acupuncture-delivery device (e.g., a wearable, external device, such as a wrist-worn device that can deliver acupuncture stimulation to the patient), a topical medication applicator, and others. In various implementations, the cardiac assessment of the patient's risk profile can be used to automatically initiate or modify an administered therapy to the patient, such as by one of the devices mentioned above.

The system 100 includes an external computing device 106, shown in FIG. 1 as a server device, which may implement portions or all of the cardiac risk assessment methods described herein, and which may communicate with the implantable device 102 wirelessly, such as over network 108. Optionally, the implantable device 102 may communicate with one or more intermediary external devices (not shown in FIG. 1), such as a handheld device, a wearable external device, or a home base station device, for example, any of which may then communicate with the external computing device 106 over the network 108.

In general, the system 100 can be used to sense and record a cardiac signal of the patient 104, and process the sensed cardiac signal to calculate sensitivity of cardiac function to sympathetic drive. In various implementations, the processing may be performed within the implantable device 102, within the external computing device 106, within an intermediary external device, or partially within two or more of the devices described above. The sensitivity may be trended over time, and the trend may be evaluated as an indicator of a degree of cardiac risk for the patient 104. Again, the trend analysis may occur within the implantable device 102, within the external computing device 106, within an intermediary external device, or according to some combination of the above.

In FIG. 1, the patient 104 is shown with the implantable device 102 implanted in a subcutaneous pocket beneath the patient's skin. In other implementations, the device 102 can be implanted such that one or more connected leads extend into the patient's heart. In still other implementations, the device 102 is an external device, such as an external ECG-sense device with electrodes for attachment to the patient's skin, or an external device for measuring an internal patient body pressure (e.g., blood pressure). As mentioned above, in some implementations the device 102 may wirelessly transmit information (either the raw cardiac signal data or information derived from processing the cardiac signal data) over the network 108, which may include various networks and devices that comprise the Internet, for receipt by the remote server 106. In some implementations, the implantable device 102 telemeters data to an intermediate external device, such as a handheld activator device or a base station unit in the patient's home, and the intermediate external device sends the data over the network 108 to the external processing equipment (e.g., server 106). Communications between any of the devices described above may be bidirectional or unidirectional, according to various implementations.

After sensing the cardiac signal data, the sensing device 102 can store a sample of the cardiac signal data in internal memory. In some implementations, the device 102 may not record until a specified triggering event occurs. In some cases, the device 102 may evaluate the sensed signal data, and may determine whether or not to store the sensed data for later processing. For example, the device 102 may determine that the sensed data is corrupted by noise to such a degree that analysis results are likely to be unduly compromised, and may accordingly choose not to store the data in internal memory. In this fashion, memory space within the implantable device 102 may be conserved.

Alternatively, the device may mark noisy data to indicate that processing of the data should be adjusted to account for the noise present with the stored data. Such noise can be caused by ectopic beats (for example, caused by premature ventricular contractions), uncorrelated beats, EMG signals, or electrical interference, to list just a few examples. In some implementations, the sensing device 102 can sense or measure the cardiac signal data and transmit the cardiac signal data without internally storing the data.

Risk assessment data may be presented to a health care professional 110, as by displaying the assessment data or information on a display screen of a monitoring device 112 at a hospital, care center, remote monitoring facility, or the like. The information may be presented in any number of ways, as will be described more fully below, and the health care professional 110 can review the information to quickly and easily assess the patient's risk of adverse cardiac events, patient's disease progression, patient compliance or noncompliance with a therapy regimen, or efficacy of the patient's treatment plan, according to various implementations. The monitoring device 112 may receive information from the external computing device 106 over a wired or wireless communication connection, including over a local area network, a wide area network, or the Internet.

The monitoring device 112 can include a program that displays the data or information from the server 106 graphically on a display device. Graphical or textual information may be presented, as well as audible or tactile information, or combinations of the foregoing, depending upon the implementation. For example, the monitoring device 112 may display a graph of trended sensitivity information, and the health care professional may interpret the data to make an assessment. Alternatively, the monitoring device 112 can display the data using numeric or text-based means, including providing a heart rate onset value associated with an occurrence of an indicator of cardiac function, such as a heart rate value above which sustained elevations of the indicator are present. In some examples, the monitoring device 112 may present statistics indicative of cardiac risk, such as a slope value or offset value (e.g., zero crossing) associated with a linear fit equation describing a relationship between parameters of cardiac function and sympathetic drive.

In some implementations, the server 106 can send an e-mail or other communication (phone call, text message, SMS message, pager signal, etc.) to the health care professional 110 when an issue arises, such as if the trending data exceeds a predetermined threshold value, or if a slope of the trending data exceeds a predetermined threshold value. Other cases that might call for physician or health care provider notification may include current or recently observed cardiac signal data indicative of an impending adverse cardiac event. In these cases, immediate medical attention may be summoned, or pre-emptive therapy measures may be initiated. Similarly, such a message may be alternatively or simultaneously communicated to the patient 104 (via e-mail, phone call, text message, SMS message, pager signal, etc.), to encourage the patient to seek medical attention or initiate medication or therapeutic measures. The monitoring device 112 may signal changes using a warning sound or a display of lights to alert the health care professional 110. The monitoring device 112 is shown as a computer (e.g., a desktop, laptop, or client-type computing device) in FIG. 1, but in some implementations the monitoring device 112 can be a hand-held or mobile device able to receive wireless communications, such as a mobile phone, smartphone, or PDA. The monitoring device 112 can also be a device worn or carried by the patient 104 (or physician, e.g.). In various implementations, the methods and procedures described herein may be practiced in a clinical setting or in an ambulatory setting, where the patient is free to go about her daily activities over an extended period of time (days, weeks, months, years, etc.), as illustratively represented by the system 100 of FIG. 1. In this fashion, the devices, systems, and methods may provide “24/7” patient risk profile monitoring and analysis over extended periods of time.

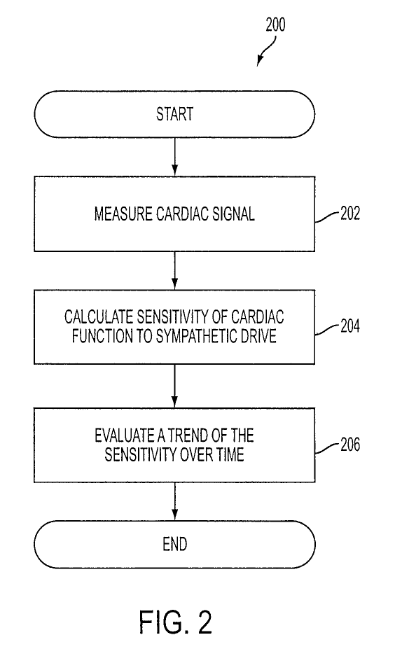

FIG. 2 is a flow chart of an exemplary process 200 that can be used to assess patient cardiac risk using cardiac signal data. At step 202, a cardiac signal is measured. The signal can be measured or received following various triggers. The cardiac signal may be a physiologic signal that is associated with the patient's cardiac cycle. The cardiac signal may be an electrical signal or a hemodynamic signal. Examples of electrical cardiac signals that can be measured include an ECG signal, an electrogram signal, or portions of such signals (e.g., the QRS complex, the repolarization wave, or subsets thereof). Such electrical cardiac signals may be measured by two or more sense electrodes within or outside the body of the patient. Examples of hemodynamic signals that can be measured include pressure signals, such as a blood pressure signal within a chamber of the heart or within the patient's cardiovascular system (e.g., within an artery or a vein), or taken using an external sense measurement (e.g., traditional arm pressure cuff), or a blood flow signal, such as a blood flow measurement taken within or across an artery or vein, for example. As another example, an electrical impedance may be measured, as by injecting a known current, measuring the resulting induced voltage (or alternatively, providing a known voltage and measuring current), and computing associated impedance according to Ohm's law. Additional examples of implantable devices capable of measuring an internal body pressure are provided in U.S. patent application Ser. No. 10/077,566, filed Feb. 15, 2002, and titled “Devices, Systems and Methods for Endocardial Pressure Measurement,” and U.S. Pat. No. 6,033,366, titled “Pressure Measurement Device,” and U.S. Pat. No. 6,296,615, titled “Catheter With Physiological Sensor,” the entire disclosures of which are herein incorporated by reference in their entirety. Additional examples of implantable devices capable of measuring an impedance are provided in U.S. patent application Ser. No. 11/933,872, filed Nov. 1, 2007, and titled “Calculating Respiration Parameters Using Impedance Plethysmography,” the entire disclosure of which is herein incorporated by reference in its entirety. Additional examples of implantable devices capable of measuring electrical cardiac signals, such as ECG signals, are provided in U.S. patent application Ser. No. 11/119,358, filed Apr. 28, 2005, and titled “Implantable Medical Devices and Related Methods,” the entire disclosure of which is herein incorporated by reference in its entirety. In some implementations, the signal may be sensed or measured and transmitted by a first device for receipt by a second device, such as over a wired or wireless communication channel.

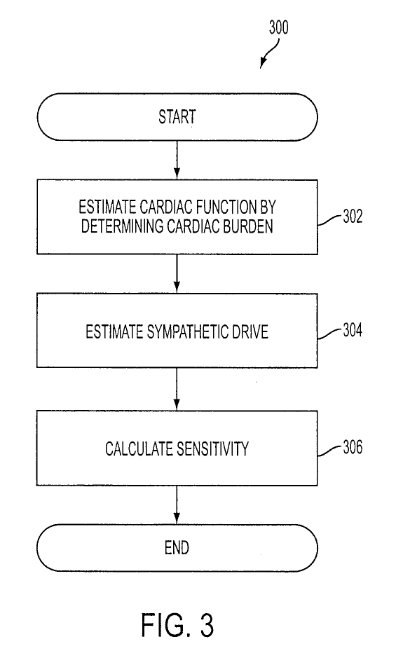

Some implementations can bin (i.e., store the information according to a particular feature) cardiac data according to various parameters. For example, the cardiac data can be binned according to time of day that the data is collected. In some implementations, measured cardiac data can be binned by heart rate associated with the data sample. In other implementations, the cardiac data can be binned according to the components of cardiac data collected, such as T-wave alternans information (or QRS or mechanical alternans information, etc.) heart rate variability information (described below with reference to FIG. 3), etc., as appropriate. In still other implementations, the cardiac data can be binned according to the type of therapy the patient is undergoing while the ECG data is collected.

Next, at step 204, sensitivity of cardiac function to sympathetic drive may be calculated or determined. The measured cardiac signal can include information from multiple cardiac cycles at multiple periods in time, and sensitivity of cardiac function to sympathetic drive can be calculated using information from several or all of cycles or periods. As will be described in more detail below, cardiac function may be indicated by an alternans burden over a period of time, or alternatively by a hypertension burden over a period of time. The alternans burden or the hypertension burden, depending on the implementation, can be determined from the measured cardiac signal.

Several types of alternans burdens can be determined. The alternans burden can be determined by analyzing the cardiac signal, or a portion of the cardiac signal, for periodic variability in the signal that may be indicative of cardiac instability. The periodic variability may be referred to as “alternans” of the physiologic signal. For example, an alternans amplitude in a 2:1 pattern (i.e., ABABAB . . . ) can be measured as a composite of amplitudes at frequencies that are odd multiples of one half of the heart rate (HR) (e.g., at 0.5*HR, 1.5*HR, 2.5*HR, etc.).

In some implementations, the physiologic or cardiac signal can be an ECG signal, or a portion of an ECG signal. For example, the method can be used to analyze an ECG signal, and specifically the T-wave or repolarization wave of the ECG signal, to determine if a patient exhibits T-wave alternans, a periodic variability associated with the T-wave of the ECG signal. In another example, the method can be used to analyze another portion of the ECG signal, such as the QRS complex, to detect QRS alternans associated with the QRS complex. In other implementations, an alternans burden associated with yet another portion of the ECG signal can be computed. As will be described further below, determination of the alternans burden may be used in assessing the patient's risk profile or to predict a patient's risk of sudden cardiac death. In other implementations, the method can be used to analyze a hemodynamic signal. Examples of methods that can be used to analyze alternans of a physiologic signal can be found in U.S. Provisional Application No. 60/991,650, filed Nov. 30, 2007, and titled “Physiologic Signal Processing To Determine A Cardiac Condition,” the contents of which are herein incorporated by reference in its entirety.

In implementations where the measured cardiac signal is a hemodynamic signal, the alternans burden may be a mechanical alternans burden determined from the hemodynamic signal. For example, in some implementations the measured cardiac signal is a blood pressure signal or a blood flow signal, either of which may be analyzed to determine a mechanical alternans burden. The mechanical alternans burden may be indicated by alternans (e.g., mechanical pulsus alternans) present in the hemodynamic signal, for example at frequencies that are odd multiples of one half of the heart rate.

In implementations where a hypertension burden is determined, the hypertension burden may be due to elevated systolic blood pressure, elevated diastolic blood pressure, or elevated systolic pressure and elevated diastolic pressure. In various implementations, pressure may be measured within a heart of the patient or outside the heart of the patient, such as within the vasculature of the patient (e.g., within an artery or vein), or even using an external pressure measurement apparatus. Also, impedance can be a surrogate for blood pressure or flow in various implementations, and a mechanical alternans burden may be determined from a signal comprised of multiple impedance measurements.

In some implementations, cardiac function can be determined from a combination of the above indications. In the example of a measured ECG signal, for example, a repolarization alternans burden and a QRS alternans burden may be determined, and combined to form an alternans burden representative of both the repolarization burden and the QRS burden. In similar fashion, in cases where the measured cardiac signal is a hemodynamic signal (e.g., a blood pressure signal), a determined mechanical altemans burden may be combined with a determined hypertension burden to form a hemodynamic burden representative of both the mechanical alternans burden and the hypertension burden. In some implementations, two or more cardiac signals (e.g., an ECG signal, blood pressure signal, blood flow signal, or signal comprised of impedance measurements) can be measured, and one or more cardiac function determinations can be made from each of the two or more cardiac signals. Without limitation, combinations can include determining a repolarization altemans burden and a mechanical alternans burden, determining a repolarization altemans burden and a hypertension burden, determining a QRS altemans burden and a mechanical alternans burden, or determining a QRS altemans burden and a hypertension burden. A hemodynamic burden determined from a measure of blood flow can similarly be used to assess cardiac function or combined with one or more of the measures discussed above.

As described above, sensitivity of cardiac function to sympathetic drive can be calculated at various periods of time. In various implementations, sympathetic drive can be estimated from a heart rate variability measurement or parameter determined from the cardiac signal. In some implementations, sympathetic drive can be estimated from a heart rate increase measurement determined from the cardiac signal. Heart rate can be tracked and trended over time, and changes in a patient's heart rate can be used to refine calculations disclosed herein. For example, in some implementations analysis may focus on periods where heart rate is increasing or accelerating, as such periods may be especially relevant for predicting patient cardiac instability. For example, a study by Narayan and Smith found that T-wave alternans (TWA) observed during periods of heart rate acceleration were more accurate in predicting ventricular tachycardia inducibility, see 35 J. Am. C. Cardiology, 1485, 1485-92. The study also showed that elevated TWA during a heart rate deceleration phase has lower predictive value, see id. In an implementation, heart rate history (e.g., acceleration and deceleration) can be tracked, and data corresponding to periods where heart rates are accelerating or decelerating can be analyzed separately, possibility using different analysis methods. For example, an altemans burden computation may be adjusted depending upon whether heart rate is accelerating or decelerating. In some cases, analysis may be adjusted during periods where heart rate is decreasing or decelerating, such as when the heart rate decrease drops below a threshold value after having been above the threshold for a predetermined time. This adjustment may account for a hysteresis effect in repolarization altemans, for example, that may occur during periods of recovery from a high-heart-rate state.

In some implementations, sensitivity of cardiac function can be calculated by computing a ratio of change in an alternans burden to a change in sympathetic marker tone. In other implementations, sensitivity of cardiac function can be calculated by computing a ratio of change in a hypertension burden to a change in sympathetic marker tone. Sympathetic tone markers and cardiac function indicators (e.g., alternans burden or hypertension burden) may be tracked, and the sensitivity of cardiac function may be calculated as an onset value of the sympathetic tone marker at which a sustained elevation in the alternans burden or hypertension burden appear. In some implementations, the onset value may be expressed as a heart rate value, but it could similarly be expressed, for example, as a percentage of the patient's maximum heart rate, or as a range of heart rate values.

The calculated sensitivity of cardiac function to sympathetic drive can be stored, and a trend of sensitivities over time can be compiled. In an implementation, the trend may include a plot of sensitivity of cardiac function to sympathetic drive versus time. In another implementation, the trend may include a plot of slope of sensitivity of cardiac function to sympathetic drive versus time.

At step 206, the trend of the sensitivity over time can be evaluated as an indicator of a degree of cardiac risk for the patient. In various implementations, the trend can be monitored and changes in the trend can be detected, which changes may indicate increased patient cardiac vulnerability in some cases. For example, the trended data may show an increasing tendency over time, and may eventually exceed a predetermined threshold level. In some implementations, slope of the data may be trended, and the slope may exceed a predetermined threshold value.

Many different methods of statistical analysis can be employed to analyze the trend information. As one example, the trend data may exceed a predetermined threshold value or the slope of the trend data may exceed a predetermined threshold value, as described above. As another example, a change can be determined if the trend meets a threshold percentage change within a predefined time period. In general, statistical analysis methods for change detection can be applied to detect change in trend.

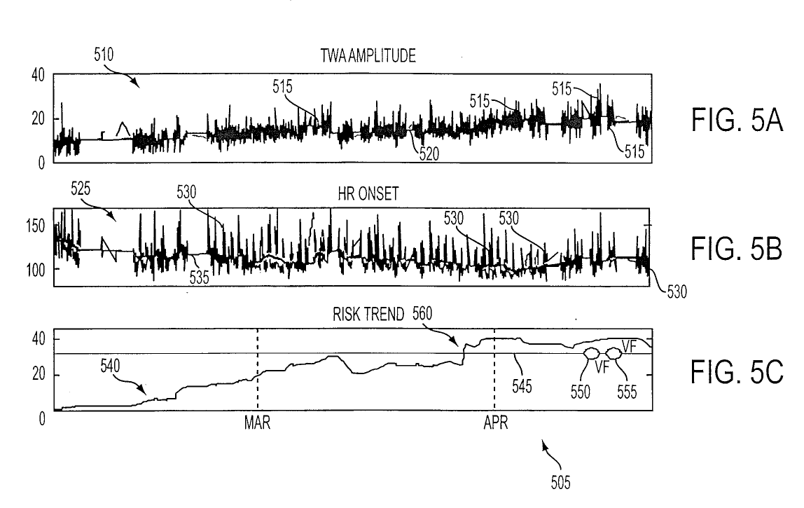

FIGS. 5A, 5B, and 5C are exemplary charts of data trended over time. The charts 5A, 5B, 5C share a common horizontal axis 505 of time listed by month, and display data covering an exemplary monitoring period from the beginning of February through the end of April, in this example. FIG. 5A illustrates an exemplary trend 510 in TWA amplitude over time, and shows T-wave alternans amplitudes 515 (the light gray signal) plotted against time, and a filtered TWA amplitude signal 520 (the black signal) superimposed over the TWA amplitude data 515. As will be described below, TWA provides one example of a cardiac burden, but any of the indicators of cardiac burden discussed herein may alternatively be trended. FIG. 5B illustrates an exemplary trend 525 in HR onset over time, and shows HR onset values 530 (the light gray signal) plotted against time, and a filtered HR onset signal 535 (the black signal) superimposed over the HR onset signal 530.

FIG. 5C trends cardiac risk 540 versus time. The cardiac risk trend 540 can be determined using data from the trended data shown in FIGS. 5A and 5B. The cardiac risk trend 540 may be determined in a variety of ways. As one example, an autoregressive model that uses a sliding window with time-varying coefficients can be used to trend the data. Changes in the coefficients can be added or summed to produce a weighted and bounded cumulative sum chart in the plot of FIG. 5C. The weights can be adjusted based on HR onset data, such as the data shown in FIG. 5B. The cumulative sum can be evaluated to detect persistent shifts in the trended signal data that may indicate changes in cardiac risk. In some implementations, evaluation to detect changes in the patient's symptoms indicated by shifts in the sensor data can involve comparing the cumulative sum to one or more thresholds, such as threshold 545. In the exemplary plot shown in FIG. 5C, an increase in cardiac risk, including an increase 560 above the threshold 545, is followed by two ventricular fibrillation episodes 550, 555. Using techniques described herein, an alert may be provided (e.g., to a health care professional or to the patient) when the cardiac risk trend meets or exceeds 560 the threshold 545, so that therapeutic interventions may be initiated or modified in an attempt to prevent sudden cardiac death, as may be caused by ventricular fibrillation episodes 550, 555.

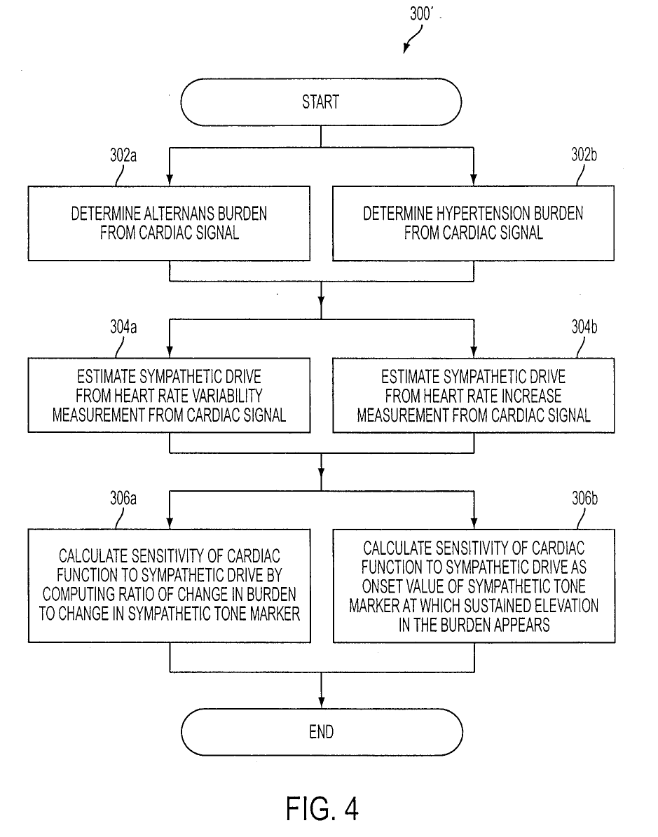

The trends shown in FIGS. 5A-C are exemplary, and variations are possible, as will be described in more detail below and with respect to FIGS. 3-4. In some implementations, trended data that exceeds a threshold may indicate that the patient's risk of an adverse cardiac event, such as sudden cardiac death, has advanced to a level where medical intervention is advisable. In these cases, for example, a physician, after observing the data or being notified of the change, may tailor appropriate medications for the patient or re-tailor the patient's therapy regimen, or suggest an ICD implant. Also, the trended information may provide visibility regarding efficacy of the patient's present therapy regimen.

In some implementations, cardiac risk can be analyzed by detecting changes in trending of autonomic parameters in relation to trend changes in cardiac function. The information can be used by health care professionals to assess cardiac risk or to determine therapy or medication effectiveness, and/or to assess patient compliance with a prescribed therapy regimen. Trending information can also be used to assess arrhythmogenic potential of new therapies and to track anti-arrhythmic remodeling effects of medications under development. As one example, some patients experience frequent arrhythmias, sometimes referred to as electrical storms. Such electrical storms may be caused by changes in autonomic balance and a vulnerable patient cardiac substrate. Using the techniques, devices, and systems described herein, relevant patient parameters may be tracked and trended for such patients, and therapy effectiveness may be evaluated in ambulatory settings and over extended periods of time. Similarly, electrical storm events may be predicted so that a preemptive therapy may be administered, as opposed to merely administering medications after the event has begun. Health care professionals can also use trending information in assessing a particular patient's cardiac vulnerability, or can aggregate such information from multiple patients for research purposes. Various autonomic parameters can be analyzed to determine different parameters of cardiac risk, including autonomic parameters detailing sympathetic and parasympathetic drives.

According to some implementations, trending information can be used to determine appropriate modifications to a patient's medication dosage or schedule. For example, a health care professional can use trending information to determine if a patient needs an up-titration in their medication. Similarly, the health care professional can use trending information to determine if a specific therapy is working for a patient, or if an alternate therapy should be considered. In other implementations, the trending information can be used to determine if a patient is adhering to dietary requirements or therapy routines. For example, a patient may not be taking a prescribed medication regularly because of unpleasant side effects caused by the prescribed medication. Trended information can provide an indication to the health care professional that the patient may not be following the prescribed therapy regimen (e.g., taking a medication at prescribed intervals and in prescribed amounts).

Trends in a cardiac burden and trends in heart rate changes can be monitored to assess therapy effectiveness in managing a patient cardiac substrate. Many medications that attempt to change a patient's arrhythmic substrate using anti-arrhythmic remodeling by impacting ion channels on a molecular level are presently under development. The techniques, devices and systems disclosed here may be useful in clinical and preclinical testing of these therapies, as the parameters affecting patient vulnerability may be monitored and assessed over extended periods of time, so that therapy effectiveness and patient vulnerability may be tracked.

FIGS. 3 and 4 are flow charts of exemplary processes 300 (FIG. 3), 300′ (FIG. 4) that can be used to calculate sensitivity of cardiac function to sympathetic drive. In general, the process 300′ of FIG. 4 provides additional detail relating to the process 300 of FIG. 3, and includes various options for performing the steps of the FIG. 3 process 300, as will be described in further detail below. In some implementations, the processes 300 and 300′ can correspond to step 204 in the process 200 of FIG. 2.

Referring first to FIG. 3, a cardiac function is estimated by determining a cardiac burden at step 302. The cardiac burden may represent an indicator of cardiac instability or patient vulnerability. In some cases, such instability may be caused by ischemia or other cardiac myopathies. Processing is performed on the measured cardiac signal, whether an electrical or hemodynamic signal. The cardiac burden may be determined over a period of time by considering strips of cardiac signal data measured periodically over the given time period (e.g., over several minutes, hours, days, weeks, months, or years). In various implementations, cardiac signal data may be measured intermittently, such as for a predetermined time (strip length) at a predetermined interval periods. Also, in some implementations cardiac signal data may be measured in response to a detected physiologic trigger, or in response to a manual trigger, as might be initiated by the patient or by a health care provider.

FIG. 4 shows that the cardiac burden may be determined from an alternans burden determined from the cardiac signal (302a), or from a hypertension burden determined from the cardiac signal (302b). For example, a repolarization alternans burden may be determined from an ECG (or electrogram) signal or from a repolarization signal comprised of extracted T-waves from the ECG signal. Without limitation, such a signal may take any number of forms, such as a continuous signal formed from the extracted T-waves, or a discrete signal, such as a matrix of successive T-waves. Similarly, a QRS burden may be determined from the ECG signal or from a QRS signal extracted from the ECG signal (e.g., continuous or discrete). In implementations where the measured cardiac signal is a hemodynamic signal (e.g., blood pressure, blood flow, or impedance), a mechanical alternans burden may be determined by detecting alternans in the signal, such as alternating high and low systolic blood pressure.

A hypertension burden may be determined from the hemodynamic cardiac signal. The hypertension burden may be due to elevated diastolic blood pressure, elevated systolic blood pressure, or a combination of the two. In various implementations, a measured impedance signal may serve as a surrogate for a blood pressure or flow signal. In this fashion, the impedance signal may be analyzed to determine an alternans burden as appropriate. In this case, cardiac risk may be evaluated by processing a surrogate for pressure without use of a pressure sensing transducer.

Next, sympathetic drive is estimated at step 304. Sympathetic drive may be estimated by considering the measured cardiac signal. As shown in FIG. 4, sympathetic drive may be estimated from a heart rate variability (HRV) measurement from the cardiac signal (304a), or from a heart rate increase measurement from the cardiac signal (304b). Sympathetic drive may be estimated so that a relationship between sympathetic drive and cardiac function may be trended and analyzed over time.

Heart rate, heart rate changes, or heart rate variability may be trended and tracked over time, and may be determined in a number of ways, according to various implementations. For example, within a given measurement strip of multiple cardiac cycles, heart rate can be determined by calculating a period between recurring periodic features of the cardiac signal, where the recurring periodic features correspond to a portion of the patient's cardiac cycle. For example, a period between successive R-waves of an ECG signal (i.e., the R-R interval) or between successive QRS complexes may be calculated. The heart rate frequency for particular cardiac data can be used to analyze multiple sets of cardiac data over time. In other implementations, heart rate variability or changes in heart rate can be determined as another autonomic parameter.

In some implementations, heart rate data can be computed over extended periods of time, and can be used to trigger data acquisition by the implantable device. In some cases, slope of the patient's trended heart rate may be used in the analysis method. For example, intervals where the patient's heart rate is accelerating, which may be indicated by an increasing slope of the trended heart rate data, may coincide with periods where an increased cardiac burden (e.g., an increased repolarization altemans burden) may indicate patient cardiac vulnerability.

Long-range heart rate variability measures can include, as one example, standard deviation of the averages of R-R intervals in multiple or all five minute segments (SDANN). In such an instance, determining trends where the SDANN increases for a patient over time can provide a health care professional with information useful in evaluating therapy efficacy or patient compliance.

Heart rate variability or changes in heart rate may serve as a surrogate for autonomic tone, according to some implementations. For example, heart rate variability can serve as an indicator of a patient's cardiac autonomic modulation. Because short-term heart rate regulation may be predominantly governed by sympathetic and parasympathetic neural activity, examining heart rate fluctuations can provide a window for observing the state and integrity of the autonomic nervous system. Long-range heart rate variability measures can provide information useful in prognostic prediction, and can include the standard deviation of the mean values of successive heart period epochs and power in very-low frequency (VLF) bands. Reductions in SDANN and VLF can indicate poor survival prospects for patients, for example, if they have chronic, severe mitral regurgitation, an acute or recent myocardial infarction, or idiopathic dilated cardiomyopathy, or have been assessed for arrhythmias, as described for example in Stefano Guzzetti et al., Different Spectral Components of 24 h Heart Rate Variability are Related to Different Modes of Death in Chronic Heart Failure, Eur. Heart J., (2005) 26: 357-62, and Serge Boveda et al., Prognostic Value of Heart Rate Variability in Time Domain Analysis in Congestive Heart Failure, J. Interventional Cardiac Electrophysiology (June, 2001) 5(2): 181-87.

In some implementations, other power spectral density parameters of HRV data may be computed. For example, power spectral density may be separated into multiple frequency zones, such as very low (e.g., below about 0.04 Hz), low (between about 0.04 Hz and 0.15 Hz), and high (between about 0.15 Hz and 0.4 Hz), see A. Malliani et al., Cardiovascular Neural Regulation Explored in the Frequency Domain, Circulation, (1991) 84: 482-92. The high frequency band is believed to be dominated by the parasympathetic nervous system, while the low frequency band is believed to be mediated by sympathetic and parasympathetic nervous outflows, see id. LF-to-HF ratios may be used to access autonomic balance as an approximation. However, recent studies suggest that the parasympathetic contributions to LF may be as significant as those of the sympathetic nervous activities; consequently, the LF-to-HF ratio may not be an accurate measure of the autonomic balance. The principal dynamic mode can be used to separate dynamics of the two nervous systems, as described in Yuru Zhong et al., Quantifying Cardiac Sympathetic and Parasympathetic Nervous Activities Using Principal Dynamic Modes Analysis of Heart Rate Variability, Am. J. Physiology Heart Circ. Physiology, (September 2006) 291:H1475-83. It is based on extracting only the intrinsic dynamic components of the signal via eigendecomposition. See id.

In some implementations, physiologic data can be combined with measuring heart rate fluctuation following a ventricular premature beat, termed here as heart rate turbulence (HRT). In various implementation, HRT may be a surrogate for baroreflex sensitivity, and may provide useful information in making the assessments discussed above. In general, baroreflex is a homeostasis mechanism that the body uses in an attempt to maintain stable blood pressure, in conjunction with the autonomic nervous system. Baroreceptors at various locations throughout the body monitor blood pressure changes, and relay the changes to the brain stem so that heart rate can be accordingly increased or decreased. Two HRT parameters of interest include onset, which can reflect an amount of sinus acceleration following a ventricular premature beat, and slope, which can reflect a rate of sinus deceleration following sinus acceleration. HRT can be used to assess a patient's cardiac risk, such as to predict long-term cardiovascular mortality for the patient abnormality.

Next, sensitivity of cardiac function to sympathetic drive is calculated at step 306. As shown in FIG. 4, sensitivity of cardiac function to sympathetic drive can by calculated by computing a ratio of change in burden to change in sympathetic tone marker (306a). Alternatively, sensitivity of cardiac function to sympathetic drive can be calculated by determining an onset value of sympathetic tone marker at which sustained elevation in the burden appears (306b). The sustained burden may be compared to a predetermined threshold level, for example, or alternatively the threshold may be updated over time based on changing circumstances. As previously described, the cardiac burden may be indicated by an alternans burden or a hypertension burden.

In various implementations, circadian variation or variability may be considered when evaluating trends of sensitivity over time. For example, with some implementations, the analysis can occur on a daily basis, such as corresponding to a particular time each day. The analysis can alternatively be performed over other time periods, such as on an hourly, weekly, or monthly basis. In some implementations, a trend may be adjusted to account for circadian variability. For example, a patient's sympathetic tone may vary substantially over the course of a day due to circadian rhythms that affect physical parameters within the body. Such impact may occur independent or semi-independent of patient activity levels, according to some implementations. By factoring circadian variability into cardiac risk assessment determinations, it may be possible to obtain more accurate results according to some implementations. In some implementations, in addition or in lieu of consideration of circadian variability over a single day, variability over two or more (three, four, five, etc.) days may be considered, and the trending information may be appropriately adjusted to account for the variability. Using the methods disclosed here, such changes may be unmasked and used to refine the analysis procedure to more accurately assess a patient's cardiac state, or assess therapy effectiveness for the patient. In other implementations, the signal may be filtered to average over circadian variability and produce a long-term trend.

As described, the methods consider a relationship over time between a cardiac burden and a heart rate parameter, as opposed to looking at a snapshot in time. The relationship is tracked and trended over time, typically in an ambulatory setting where the patient is free to go about their daily activities without the inconvenience of arranging and visiting a clinical facility for dedicated testing during a condensed time period. As such, the results obtained using the methods disclosed here may provide more accurate assessment data in some implementations, and may be more convenient for the patient.

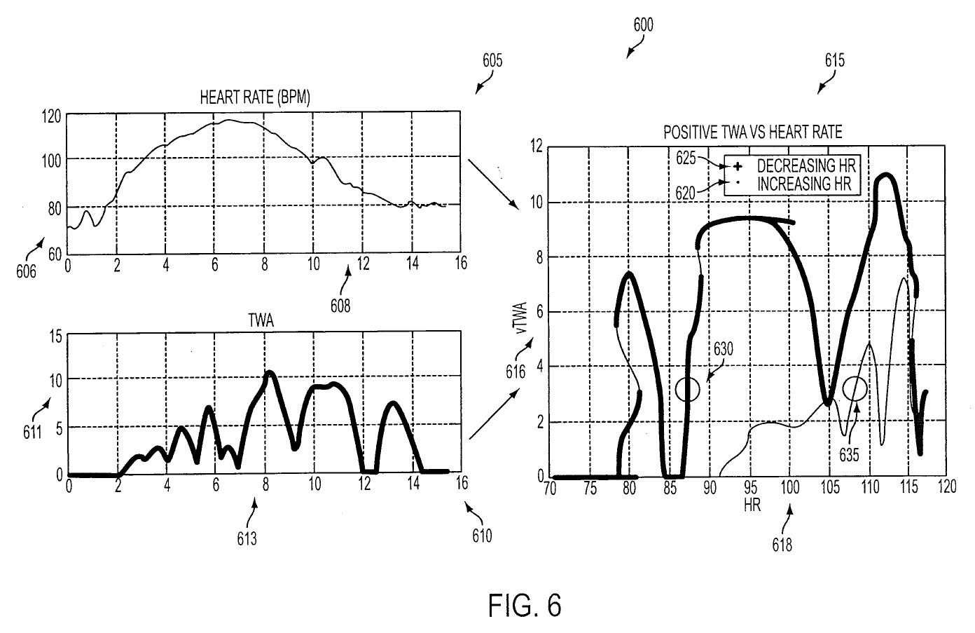

FIG. 6 is a series of exemplary charts 600 that can be used to assess a patient's cardiac risk profile. A first chart 605 shows heart rate changes over a period of time by plotting heart rate versus time, using a vertical axis 606 of heart beats per minute and a horizontal axis 608 of minutes. Heart rate can be measured periodically over the given time interval, according to various implementations. A second chart 610 shows T-wave altemans amplitude over the same time period, using a vertical axis 611 of microvolts and a horizontal axis 613 of minutes. In other implementations, one or more different indicators of cardiac burden, such as QRS alternans, mechanical alternans from a hemodynamic signal, or a hypertension burden, may be alternatively substituted or used in conjunction with the trend of TWA 610. As can be seen with reference to the first and second charts 605, 610, TWA generally have higher amplitude during periods of higher heart rates. Higher heart rates may indicate increased sympathetic tone, and the calcium ions that mediate the propagation of the heart's electrical signals may not have time to fully cycle at high heart rates, which can lead to alternans in the T-wave of the ECG.

A third chart 615 combines information from the first and second charts 605, 610, to display TWA amplitude versus heart rate. As such, the third chart provides information on the relationship between TWA and heart rate, so that the relationship may be trended over time, and changes in the trend may be tracked and correlated to patient progress or vulnerability. A vertical axis 616 has units of microvolts, while a horizontal axis 618 has units of heart beats per minute. Additionally, the plot uses line width to depict periods where the patient's heart rate is increasing (narrow line width 620) or decreasing (wider line width 625). The charts in the series 600 show data collected over a short time period, but in other implementations the data may be collected over one or more days, weeks, months, or years, and the data may be trended in similar fashion.

The third chart 615 provides an example of an indicator of cardiac function (the TWA in this case) versus a measure of sympathetic drive (heart rate in this case). A trend of the sensitivity of the cardiac function to sympathetic drive may be evaluated for risk stratification purposes, or to assess a patient therapy or evaluate patient compliance with a therapy regimen. In some implementations, an indicator of a degree of cardiac risk can be determined using the chart 615, such as by determining an onset value of a sympathetic tone marker.

The onset value may correspond to a sympathetic tone marker value at which a sustained elevation in the cardiac burden appears. In this case, a heart rate at which sustained increase in TWA amplitudes appear. For a given cardiac burden threshold, the onset value may be the lowest sympathetic drive measure at which sustained burden occurs, such that at sympathetic drives above the onset value, cardiac burden is measured above a threshold value for at least a predetermined period or percentage of time. T-wave altemans can be considered significant at specific voltage levels, and the voltage levels may be varied to account for numerous factors, according to some implementations. In some implementations, T-wave altemans can be considered significant above about 1.9 microvolts, although other threshold levels can be used in other implementations (e.g., about 3 microvolts in the FIG. 6 series of charts 600). For example, a first circled portion 630 may correspond to a first onset value of about 87 beats per minute for decreasing heart rate, and a second circled portion 635 may correspond to a second onset value of about 107 beats per minute for increasing heart rate in this example. In some implementations, a single onset value may be calculated that does not distinguish between periods of increasing and decreasing heart rate. In some cases, circadian variability factors can be used in determining appropriate thresholds, and the thresholds can be adjusted depending on time of day or other circadian factors.

It has been demonstrated that onset HR over which significant TWA occur is higher in healthy controls than in high-risk patients. For example, it has been demonstrated that that at higher heart rates, TWA becomes a more sensitive but less specific test for cardiac risk, see Neal Kavesh et al., Effect of Heart Rate on T wave Alternans, J. Cardiovascular Electrophysiology, (1998) 9:703-08.

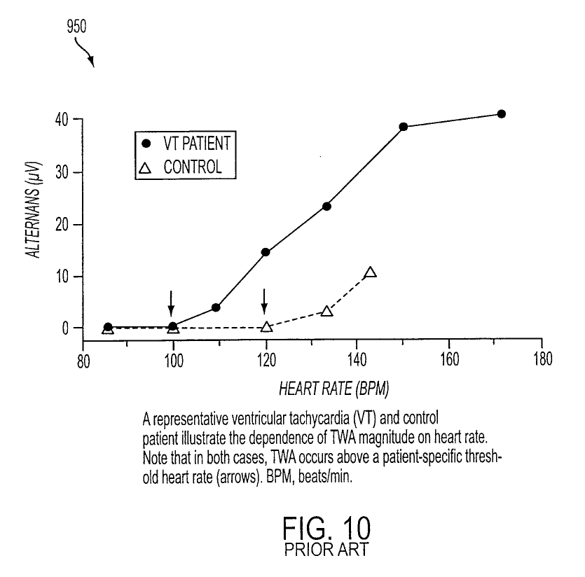

FIG. 10 is a graph 950 of an exemplary dependence of T-wave altemans on heart rate. The figure, from Elizabeth Kaufmann et al., Influence of Heart Rate and Sympathetic Stimulation on Arrhythmogenic T wave Alternans, Am. J. Physiology Heart Circulatory Physiology, (2000) 279: H1248-55, shows a graph 950 that illustrates a typical dependence of TWA amplitude on heart rate, and shows altemans amplitude versus heart rate for a representative ventricular tachycardia patient and a control (healthy) patient. As can be seen in FIG. 10, even the healthy patient exhibits altemans at elevated heart rates (e.g., above 120 bpm in this example), while the diseased patient exhibits alternans at lower heart rates (and with higher amplitudes in this example). The concept of an onset heart rate value, which may be defined as a heart rate value at which sustained elevation in an alternans burden appears, is also illustrated. Graph 950 shows exemplary onset values of 100 bpm for the VT patient and 120 bpm for the control patient (each indicated by arrows in FIG. 10).

As described above, patients may exhibit higher levels of TWA during periods of decreasing heart rate, especially when such periods follow periods of high heart rate. This can be seen in the chart 615, and may be caused by the residual sympathetic drive after the exercise is over.

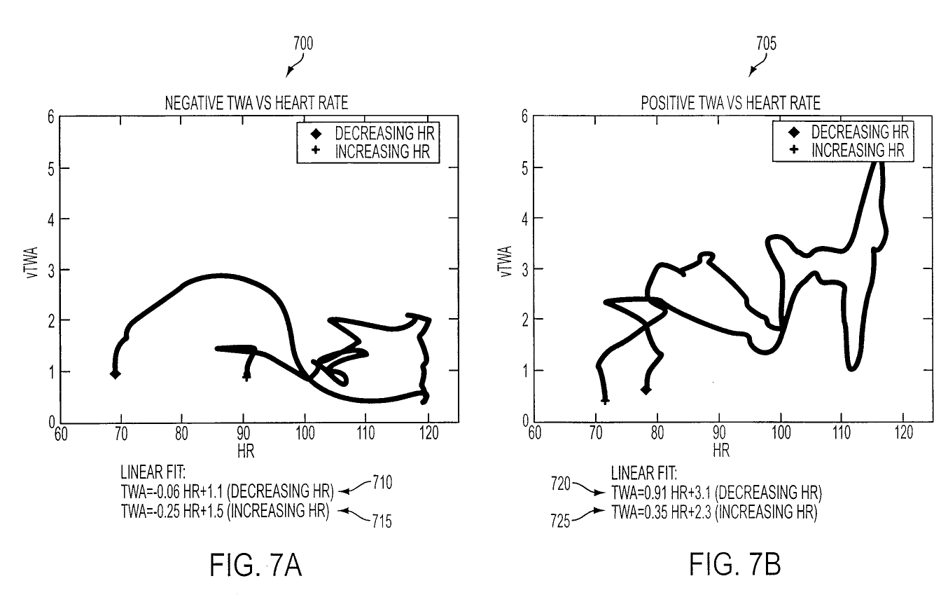

A risk assessment indicator of patient cardiac vulnerability can be provided in various ways. For example, heart rate onset data can be trended over time, as described above with respect to FIG. 5B, and a risk trend can be provided as described above with respect to FIG. 5C. In this way, a relation of a cardiac burden to sympathetic drive may be displayed and presented, such as to a health care professional. Similarly, the health care professional can be alerted through specific highlighting on a graphical display, such as a trend exceeding a threshold. The highlighting may indicate a change in the trend data relevant to patient vulnerability. For example, if the trended data eclipses a predetermined threshold, data exceeding the threshold can be identified in red or bold (or the area of the graph where the data is plotted may be highlighted, etc.). In other implementations, the health care professional can be alerted by sending a message via e-mail, text message, or phone call containing the trend information. Alternatively, an audible alarm may be sounded to correspond to certain changes in trended data. For example, if the trended data indicates a likelihood of a major cardiac event in the near future, a monitoring device can signal the health care professional or patient so that a therapy may be administered to counteract the trend change, or so that immediate medical attention may be sought. Additional risk assessment indicators are discussed below with reference to FIG. 7.

As discussed previously, various autonomic parameters can provide information relevant in analyzing a patient's cardiac state. In some implementations, heart rate variability or heart rate changes can be used to measure vagal and sympathetic modulation on heart rate. In several disease states, such as diabetes, congestive heart failure, after myocardial infarction, or after a heart transplant, heart rate variability may decrease due to vagal withdrawal and sympathetic overstimulation.

In some implementations, heart rate turbulence can be used as a predictor of long-term cardiovascular mortality in patients with previous myocardial infarctions. Heart rate turbulence can be quantified by two parameters: turbulence onset and turbulence slope. Turbulence onset can reflect the amount of sinus acceleration following a ventricular premature beat. Turbulence slope can reflect the rate of sinus deceleration that follows sinus acceleration.

FIGS. 7A and 7B are exemplary charts 700, 705 of T-wave alternans amplitude data versus heart rate for a TWA-negative patient, and TWA-positive patient, respectively. Similar to the third chart 615 in FIG. 6, the charts 700, 705 include vertical axes with units of microvolts, and horizontal axes with units of heart beats per minute. Also similarly, line style is used to differentiate between periods of increasing heart rate and decreasing heart rate. As described above, such representations 700, 705 may describe a sensitivity of cardiac function to sympathetic drive, and the data associated with the representations may be analyzed to determine one or more equations descriptive of the data. For example, a linear fit equation of the form “Y=mX+b” may be calculated, where “Y” corresponds to the cardiac function indicator (TWA amplitude in the FIGS. 7A and 7B examples); “m” corresponds to a slope of the data; “X” corresponds to the sympathetic drive indicator (heart rate in the FIGS. 7A and 7B examples); and “b” corresponds to an offset of the sympathetic drive indicator. The cardiac function sensitivity to HR can be expressed with the slope “m,” which corresponds to the ratio of the TWA change to HR change. A higher slope can indicate higher sensitivity to HR, and thus indicate higher cardiovascular risk.

In some implementations, more than one equation may be derived, where each of the more than one equations corresponds to a portion of the data under analysis. As one example, heart rate changes may be tracked, and data associated with an increasing heart rate may be binned separately from data associated with a decreasing heart rate. In these cases, data associated with a constant or minimally changing heart rate may be included with either the increasing HR data or the decreasing HR data, may be separately analyzed as a third category, or may be dropped, depending on the implementation.

A first equation 710 describes a portion of the data associated with chart 700 where heart rate is decreasing, corresponding to the chart segments having narrow line width. In this example, the equation 710 is “TWA=−0.06 HR+1.1.” Similarly, a second equation 715 describes the portion of the data associated with chart 700 where heart rate is increasing, corresponding to the chart segments having wider line width. In this example, the equation 715 is “TWA=−0.25 HR+1.5.” Equations 720 and 730 similarly describe the data plotted in chart 705. As shown, slope values (−0.06 and −0.25) from the equations 710, 715 corresponding to the TWA-negative patient are smaller than slope values (0.91 and 0.35) from the equations 720, 725 corresponding to the TWA-positive patient.

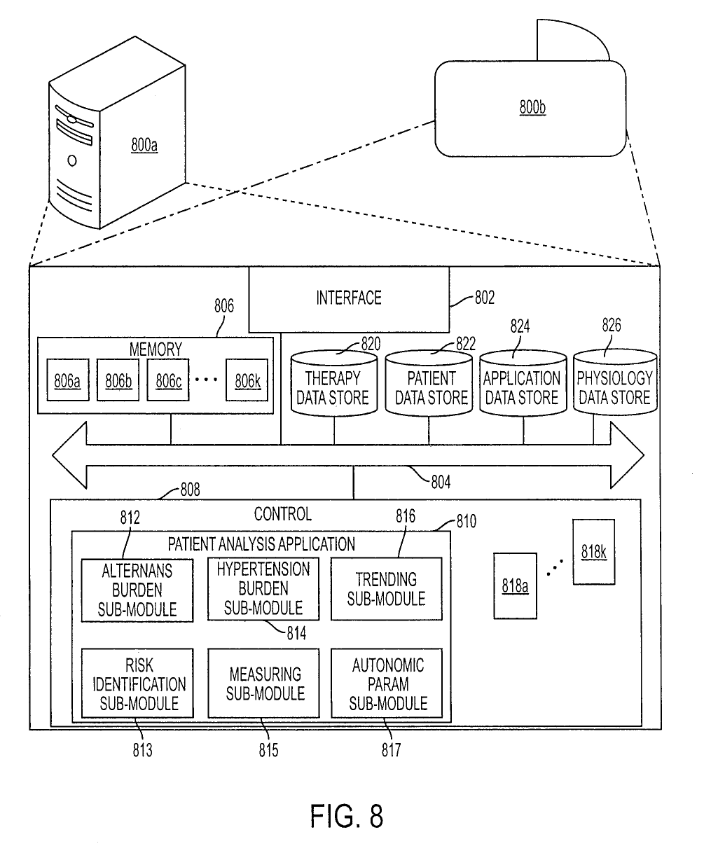

FIG. 8 is a block diagram of exemplary devices 800 that can be used to calculate sensitivity of cardiac function to sympathetic drive and evaluate a trend of the sensitivity over time. FIG. 8 shows two examples of devices 800 that can implement all or of portion of the techniques disclosed herein: a server device 800a and an implantable device 800b. In other implementations the device 800 may be another type of external computing device, such as a handheld computing device (e.g., PDA, mobile phone, smartphone, etc.) or a personal computer. In general, the devices 800a or 800b receive cardiac signal data, calculate sensitivity of cardiac function to sympathetic drive, and evaluate a trend of the sensitivity over time. In some cases, an additional step of measuring the cardiac signal may be included, such as if the method is implemented on the implanted device 800b. In some implementations, the device 800 can transfer data to a monitoring device so that a health care professional can view patient trending information and other patient-related data. The device depictions 800 are exemplary, and in various implementations the devices 800a, 800b will include only a subset of the internal components pictured. Similarly, the implantable device 800b may take different form factors, and may include various sensors or sense electrodes for measuring a cardiac or cardiovascular signal. For example, the implantable device 800b may include one or more sense electrodes on an exterior surface of the device 800b, or a sense port (e.g., a pressure sense port) on an exterior surface of the device 800b. Similarly, the implantable device 800b may include one or more leads (e.g., a subcutaneous lead or an intracardiac lead) or pressure sense catheters that may include various electrodes or sensors for measuring physiologic signals.

Cardiac signal data can be received by the device 800 through an interface 802. In implementations including external devices, the interface 802 may receive data over a communication channel or over a network, for example. In implementations implementing the techniques within implanted devices, the interface 802 may receive sensed signal readings, such as from connected electrodes or other types of sensors (e.g., a pressure sensor or blood flow sensor). In some implementations, the interface 802 includes a telemetry component that may be able to transmit or receive data wirelessly over an antenna (not shown in FIG. 8). The interface 802 can place the cardiac signal data on a bus 804, which provides interconnectivity between the various modules and components of the device 800. A control module 808 may include hardware and software modules, including one or more processors (not shown) that may execute instructions to perform tasks for the system, such as the steps comprising the methods disclosed herein. Examples of processors that may be suitable can include one or more microcontrollers, microprocessors, central processing units (CPUs), computational cores instantiated within a programmable device or ASIC, and the like. In general, the processor or other control components of the control module 808 may control or manage the flow of information throughout the system, including the flow of information over the bus 804. As is conventional, instructions and data may be stored in a non-volatile data store, and may be moved to a memory 806 for active use. In some implementations, the memory 806 can store the cardiac signal data within bins 806a through 806k. The processor may access instructions and data from memory for execution, for example, and may load the instructions and data into on-chip cache, if equipped and as appropriate.

The control module 808 includes a patient analysis application 810, which can be used to implement the trending techniques discussed herein. The patient analysis application 810 includes an alternans burden sub-module 812, a hypertension burden sub-module 814, a risk identification sub-module 815, and a trending sub-module 816, each of which may implement portions of the techniques discussed herein. Some implementations (e.g., implementations where the device 800 is an implantable device or an external monitoring device) include a measuring sub-module 817, which may be used control measurement of one or more cardiac or cardiovascular signals. The control module 808 can also optionally include other applications 818a through 818k, which may be used to perform other tasks associated with the device. For example, one of these applications may notify the patient to take specific medications as prescribed by her health care professional at particular times.

The control module 808 may request data from various data stores 820, 822, 824, 826, any or all of which may be omitted in various implementations. For example, a therapy data store 820 may store data relating to patient therapies, a patient data store 822 may store patient-specific information, an application data store 824 may store information relating to graphically displaying trending information, and a physiology data store 826 may store medical information relating to possible medical conditions. The control module 808 can process cardiac signal data and pass the processed data or information derived from analysis of the data to the interface 802 over the bus 804. From there, the interface 802 may forward the data, for example, to a monitoring device for review by a health care provider.

The device 800 can receive, process, and transmit information regarding assessment of a patient's cardiac risk profile, treatment plan efficacy, or compliance with a therapy regimen. The control module 808 can execute instructions that cause the module to receive data and determine changes in trending of autonomic parameters in a patient. For example, the control module 808 can use the patient analysis application 810 to determine trending information by detecting changes in trends of autonomic parameters.

In some implementations, the altemans burden sub-module 812 can determine altemans information from a cardiac signal. For example, if the signal is an ECG signal, the alternans burden sub-module 812 can determine T-wave alternans information. In some implementations, the alternans burden sub-module 812 can use harmonic decomposition to analyze the cardiac signal using efficient, time-frequency analysis techniques. Examples of techniques that can be used to determine alternans information from a cardiac or cardiovascular signal can be found in U.S. Provisional Application No. 60/991,650, referred to previously above. In other implementations, the altemans burden sub-module 812 can use time domain analysis to determine altemans information. In still other implementations, the altemans burden sub-module 812 can use frequency domain analysis to determine alternans information. The hypertension burden sub-module 814 can determine a hypertension burden from the cardiac signal. The hypertension burden may be due to elevated systolic pressure, elevated diastolic pressure, or a combination of both.

In some implementations, the autonomic parameter sub-module 817 can determine various autonomic parameter information from the cardiac signal data. In some implementations, the autonomic parameter sub-module 514 can determine heart rate information, such as heart rate variability information or heart rate change information. Other autonomic parameters that can be determined, with some implementations, include heart rate turbulence and/or deceleration capacity. In some implementations, indicators of sympathetic drive may be determined from the cardiac signal. Heart rate may be determined, and heart rate changes may be tracked, including distinguishing between periods of increasing heart rate and periods of decreasing heart rate. Periods may be further distinguished based on heart rate rate-of-change, or slope. In various implementations, cardiac signal data, or information derived from the data, may be stored according to these distinctions, which may facilitate improved patient analysis.

The trending sub-module 817 may calculate a sensitivity of cardiac function to sympathetic drive for data corresponding to multiple cardiac strips measured at multiple periods of time, such as over multiple minutes, hours, days, weeks, or years. These calculations may use information from the alternans burden sub-module 812, the hypertension burden sub-module 814, and the autonomic parameter sub-module 817. Trending steps may include trending an alternans burden (TWA, QRS, or mechanical alternans, e.g.), hypertension burden, heart rate, heart rate slope, HRV, HRT, deceleration capacity, baroreflex sensitivity, or relationships among two or more of the foregoing. The risk identification sub-module 813 can then evaluate a trend of the sensitivity over time as an indicator of a degree of cardiac risk for the patient. The module 813 can also assess efficacy of a patient's prescribed therapy, and/or determine likelihood of patient full-compliance with a prescribed therapy regimen, according to some implementations. In some cases, circadian variability factors can be used to adjust the trended information, or used to adjust risk identification results in view of the trended information. The circadian variability analysis may consider that at certain times of the day, sympathetic drive may tend to be higher than at other times of the day, independent of present patient activity.

In some implementations, one or more of other applications 818a through 818k can compile a display of the trending information. In some implementations, the control module 808 can receive information from other sources and apply the information to calculations in the patient analysis application 810.

Information applied to calculations determined in the patient analysis application 810 can include data from various data stores within the device 800. Example data stores can include the therapy data store 820, the patient data store 822, the application data store 824, and the physiology data store 826. These data stores can store information that can be used in assessing a patient's cardiac risk profile, therapy effectiveness, or patient compliance with a therapy regimen. In some cases, the information may be received from a health care professional, for example. The data stores can also store information received from a patient. The data stores can be updated on a periodic basis. In the depicted implementation, the data stores 820-826 reside within the device 800, but in other implementations one or more of the data stores, or other data stores storing other relevant information, may be external to the device, and may be accessed by the device 800 or by another device that provides the information to the device 800.

The therapy data store 820 can store information regarding patient therapy methods, such a drug pump, electro acupuncture therapy to induce parasympathetic activation, a cardiac rhythm management device, topical medication applicators (e.g., a patch to release a topical anaesthesia), or oral medications. For example, this information can provide data to the control module 808, to additionally be used in evaluating trending information.

Information regarding the patient can also aid in determining patient cardiac risk. The patient data store 822 can include patient information such as drug allergies, previous cardiac history, and current or historical health care providers. The control module 808 can use information from the patient data store 822 to modify an assessment of trending information. The application data store 824 can be used to store information for any of the applications that may run on the device 800, of that may be used for communicating with other devices. For example, the application data store 824 may contain libraries of information that various applications may use in operation.

The physiology data store 826 can also provide information to aid in determining cardiac state by including the patient's physical data. The physiology data store 826 can include patient vital signs from previous visits, or other risk markers determined with external or implantable devices, such as a burden of non-sustained ventricular tachycardia, and other pre-existing conditions like genetic predisposition to cardiac disease, to list just a few examples. This data can be used with the techniques described here to provide a more accurate risk assessment, according to some implementations. In some implementations, the device 800a may correspond to the external computing device 106 shown in FIG. 1, and the device 800b may correspond to the implantable device 102 from the same figure.

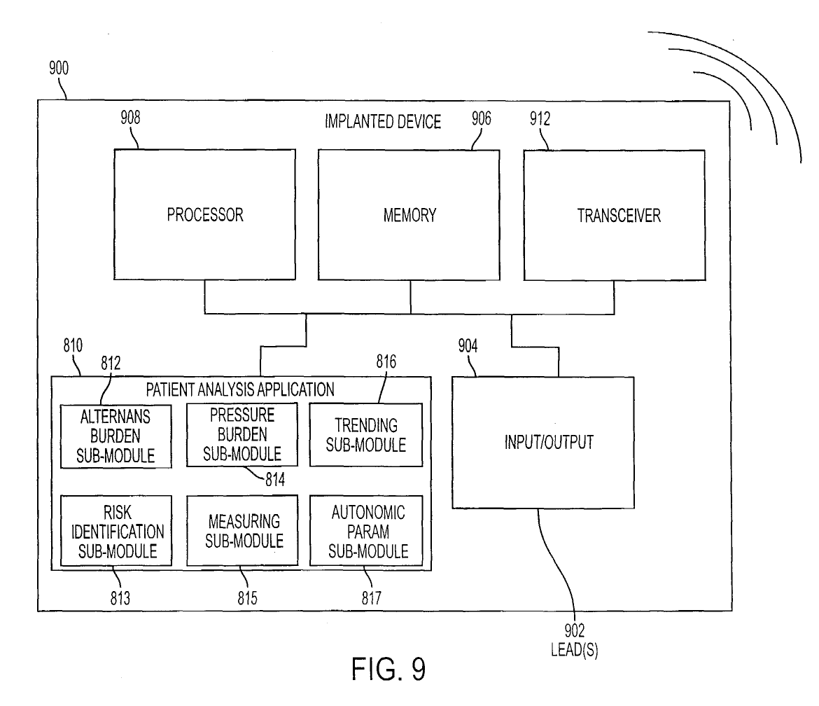

FIG. 9 is a simplified block diagram of an exemplary implantable device 900. In some implementations, the device 900 can implement any of the methods described herein, or any portion of the methods. In some cases, the device 900 can cooperate with one or more other devices (whether implanted or external) to implement the methods discussed herein. While not shown here for simplicity, in general the device 900 may include some or all of the components discussed above with reference to the device 800 of FIG. 8. In some implementations, the device 900 can implement a portion of some of the methods described herein (e.g., the device may measure and sense a cardiac or cardiovascular signal, store the signal, and wirelessly transmit the signal or a portion of the signal to an external device for further processing, without performing cardiac burden analysis, sympathetic drive analysis, trending analysis, or risk assessment). Implementations of the device 900 can be used to accurately measure risk indicator assessment over a period of time. In general, the implanted device 900 records and processes a patient's cardiac signal data. For example, the implanted device 900 can record an ECG signal, an impedance signal, or a hemodynamic signal. In some implementations, the device may sense and record multiple cardiac or cardiovascular signals, such as both an electrical signal and a hemodynamic signal, either or both of which may be analyzed (independently or cooperatively) with various autonomic parameters to determine cardiac risk.

The depicted device 900 includes one or more leads 902, which may be configured for positioning inside or outside of a patient's heart or other bodily organ, depending on the implementation, an input/output module 904, a memory 906, a processor 908, a transceiver 912, and the patient analysis application 810, described above with reference to FIG. 8, pictured stored in a non-volatile memory medium. The transceiver 912 may include a transmitter and a receiver, and may communicate wirelessly with an external (or implanted) device using an antenna (not shown). In some implementations, the implanted device 900 can incorporate the transceiver 912 and the input/output module 904 into the same module.

In some implementations, the transceiver 912 can be configured to receive command signals. For example, the receiver 912 can receive a command that instructs the device 900 to record a segment of cardiac signal data. In other implementations, the transceiver 912 can receive a command signal indicating that the implanted device 900 should record and transmit a segment of the cardiac signal data on a specified periodic basis, such as hourly, daily, or weekly. In other implementations, the transceiver 912 can receive a command signal that instructs the device 900 to measure a cardiac signal and transmit the signal without storing it within the device 900, including continuous measurement and transmit in some implementations.

The one or more leads 902 can include one or more electrodes that can sense signal data, including cardiac signal data of the patient. In some implementations, the one or more leads 902 are intracardiac leads; in some implementations, the one or more leads 902 are configured for subcutaneous positioning within a patient; in some implementations, at least one intracardiac lead and at least one subcutaneous lead are included. In some implementations, the one or more leads 902 may be replaced or supplemented with one or more sensors or ports configured to sense a hemodynamic signal. Some implementations may include one or more catheters that may facilitate hemodynamic measurements at a distance from the device. For example, a pressure transmission catheter may be used to sense a body pressure and refer the pressure to a pressure transducer, which may be housed within the body of the device 900 or in a separate housing, in which case the pressure information may be communicated to the device 900 by wired or wireless communication link. Various combinations of leads and electrodes are possible. As one example, the device may include a single lead 802 with a single electrode, and may include a second electrode on the housing 812. As another example, a lead may include two or more electrodes, or the housing may include two or more electrodes. Leadless implantable devices are also contemplated, where an exterior surface or the device includes electrodes and/or sensor(s) to make the measurements discussed herein.

In general, the processor 908 can execute instructions to perform the methods described herein. For example, the patient analysis application 810, and its various sub-modules 812-817 may include instructions that when executed perform one or more of the methods discussed herein. The patient analysis application 810 may be stored in a non-volatile medium (e.g., EPROM, flash memory, EEPROM, or various other non-volatile storage mediums familiar to those skilled in the art) within the device 900, and may be transferred to memory 906 (e.g., SRAM, DRAM, SDRAM, or various other volatile or non-volatile storage mediums familiar to those skilled in the art) for active use by the processor 908. Additional device components, such as a battery, signal processing circuitry, clocking circuitry, and optionally any of the components depicted in FIG. 8 are omitted from FIG. 9 for simplicity. In some implementations, the memory 906 and processor 908 may be implemented in a programmable device, such as a programmable logical device (PLD, e.g. an FPGA) or application specific integrated circuit (ASIC). In some applications, the patient analysis application 810 may include only a subset of the depicted sub-modules, such as only the measuring sub-module 815. In these implementations, for example, processing of the cardiac signal data may occur outside of the implantable device 900, such as in the external computing device 106 shown in FIG. 1.

Cardiac signal data may be recorded over a predetermined number of heartbeats, or for a predetermined time interval. Each recording of cardiac signal data may be referred to as a “strip” of data. In some implementations, the strips can contain data associated with 128 heartbeats, but strip lengths of any appropriate number of beats (e.g., 32, 64, 256, 512, etc.) or time period can be used. In some implementations, the implanted device 900 can make determinations using the cardiac signal data to decide whether to continue recording.

A number of implementations of the invention have been described. Nevertheless, it will be understood that various modifications may be made without departing from the spirit and scope of the methods, systems, and devices described herein. For example, the sensing device may be a stationary device, according to some implementations. The sensing device may be used in implementations where data is collected in a health care facility, such as a hospital. The sensing device can transmit data to be analyzed using the techniques disclosed herein. In implementations that use an implantable device, the implanted device may comprise two or more implantable enclosures. In some implementations, a risk indicator value derived from an electrical signal may be used in combination with a risk indicator value derived from a hemodynamic signal to assess a patient's cardiac state, or a single risk indicator value may be computed using both an electrical cardiac signal and a hemodynamic cardiac signal. Accordingly, other embodiments are within the scope of the following claims.

What is claimed is:

1. A system for assessing cardiac risk of a patient, the system comprising:
a measuring component that measures a cardiac signal comprised of multiple cardiac cycles at multiple periods in time;
a processing component that calculates sensitivity of cardiac function to sympathetic drive at each of the periods in time from the measured cardiac signal; and
a risk identification component that evaluates a trend of the sensitivity over time as an indicator of a degree of cardiac risk.

2. The system of claim 1, wherein the risk identification component evaluates the trend by evaluating a plot of the sensitivity versus time.

3. The system of claim 1, wherein the risk identification component evaluates the trend by comparing the trend with a predetermined threshold.

4. The system of claim 1, wherein the measuring component, the processing component, and the risk identification component reside in an implantable device.

5. The system of claim 1, wherein the measuring component resides in an implantable device, and wherein the processing component and the risk identification component reside in an external device.

6. The system of claim 5, further comprising a first telemetry component that resides in the implantable device and a second telemetry component that resides in the external device, wherein the first telemetry component transmits at least a portion of the cardiac signal for receipt by the second telemetry component.

7. The system of claim 1, wherein the cardiac function is indicated by an altemans burden over a selected period of time, the alternans burden determined from the cardiac signal.

8. The system of claim 7, wherein the alternans burden is a repolarization altemans burden.

9. The system of claim 7, wherein the alternans burden is a mechanical altemans burden.

10. The system of claim 7, wherein the alternans burden is a QRS altemans burden.

11. The system of claim 7, wherein the sensitivity of cardiac function is calculated by computing a ratio of change in the altemans burden to change in a sympathetic tone marker.

12. The system of claim 7, wherein the sensitivity of cardiac function is calculated as an onset value of a sympathetic tone marker at which a sustained elevation in the altemans burden appears.

13. The system of claim 1, wherein the cardiac function is indicated by a hypertension burden over a selected period of time, the hypertension burden determined from the cardiac signal.

14. The system of claim 13 wherein the hypertension burden is elevated diastolic blood pressure.

15. The system of claim 13 wherein the hypertension burden is elevated systolic blood pressure.

16. The system of claim 1, wherein sympathetic drive is estimated from a heart rate variability measurement determined from the cardiac signal.

17. The system of claim 1, wherein sympathetic drive is estimated from a heart rate increase measurement determined from the cardiac signal.

18. The system of claim 1, wherein the cardiac signal is an electrocardiogram signal.

19. The system of claim 1, wherein the cardiac signal is an electrogram signal measured using an intracardiac lead.

20. The system of claim 1, wherein the cardiac signal is a blood pressure signal.

21. The system of claim 1, wherein the cardiac signal is an impedance measurement.

22. The system of claim 1, wherein the processing component further calculates a baroreflex sensitivity value from the measured cardiac signal.

23. The system of claim 22, wherein an indication of heart rate turbulence, determined from the cardiac signal, indicates the baroreflex sensitivity value.

24. The system of claim 23, wherein the indication of heart rate turbulence is a turbulence slope value.

25. The system of claim 23, wherein the indication of heart rate turbulence is a turbulence onset value.

26. The system of claim 1, wherein the risk identification component adjusts the trend based on circadian variability

27. The system of claim 1, wherein the risk identification component filters the trend to remove circadian variability and short term variability.

28. The system of claim 1, wherein the trend of the sensitivity over time indicates a degree of cardiac risk that exceeds an expected degree of cardiac risk because of patient non-compliance with a prescribed therapy.

29. The system of claim 1, wherein the trend of the sensitivity over time indicates a degree of cardiac risk that exceeds an expected degree of cardiac risk because of insufficient therapy effectiveness.

30. A method of monitoring effect of a cardiovascular therapy for a patient; the method comprising:
receiving physiologic signal data associated with multiple cardiac cycles;
calculating sensitivity of cardiovascular function to sympathetic drive using the received physiologic signal data;
trending the computed sensitivity over time; and
comparing the sensitivity trend to an expected trend, the expected trend based on a previous condition of the patient and a prescribed therapy regimen that specifies the cardiovascular therapy.

31. The method of claim 30, wherein the cardiovascular therapy modulates the effect of the sympathetic tone on cardiac function.

32. The method of claim 30, wherein the cardiovascular therapy modulates vascular tone.
